(19) United States
(12) Patent Application Publication
(10) Pub. No.: US 2004/0029128 A1
(43) Pub. Date: Feb. 12, 2004

(54) Methods and nucleic acids for the analysis of CpG dinucleotide methylation status associated with the calcitonin gene

(75) Inventors: Susan Cottrell, Seattle, WA (US); Suzanne Mooney, Seattle, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics, Inc.

(21) Appl. No.: 10/281,076

(22) Filed: Oct. 25, 2002

Related U.S. Application Data
(63) Continuation-in-part of application No. 10/215,890, filed on Aug. 8, 2002.

(51) Int. Cl. C12Q 1/68; C07H 21/04

(52) U.S. Cl. 435/6; 536/24.3

(57) ABSTRACT

The disclosed invention provides methods and sequences for the analysis of methylation patterns within a novel 5′ upstream CpG island of the calcitonin gene. Particular embodiments provide methylation-altered DNA sequences as novel diagnostic, prognostic and therapeutic markers for cancer.

### FIELD OF THE INVENTION

The present invention relates to human DNA sequences that exhibit altered methylation patterns (hypermethylation or hypomethylation) in cancer patients. These novel methylation-altered DNA sequences are useful as diagnostic, prognostic and therapeutic markers for human cancer.

### BACKGROUND

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

Current use of bisulfite modification to assess CpG methylation status. A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, et al., A modified and improved method for bisulphite based cytosine methylation analysis, Nucleic Acids Res. 24:5064-6, 1996). Using this method, it is possible to analyze individual cells, which illustrates the potential of the method. However, currently only individual regions of a length of up to approximately 3000 base pairs are analyzed, and a global analysis of cells for thousands of possible methylation events is not possible. Moreover, this method cannot reliably analyze very small fragments from small sample quantities. Such fragments are lost through the matrix despite the diffusion protection. An overview of art-recognized methods for detecting 5-methylcytosine is provided by Rein, T., et al., Nucleic Acids Res., 26:2255, 1998.

Currently, barring few exceptions (e.g., Zeschnigk M, et al., Eur J Hum Genet. 5:9498, 1997) the bisulfite technique is only used in research. In all instances, short, specific fragments of a known gene are amplified subsequent to a bisulfite treatment and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyze individual cytosine positions, or treated by enzymatic digestion (Xiong & Laird, Nucleic Acids Res., 25:2532-4, 1997). Additionally, detection by hybridization has also been described (Olek et al., WO 99/28498).

Further publications dealing with the use of the bisulfite technique for methylation detection in individual genes are: Grigg & Clark, Bioessays, 16:431-6, 1994; Zeschnigk M, et al., Hum Mol Genet., 6:387-95, 1997; Feil R, et al., Nucleic Acids Res., 22(4):695-, 1994; Martin V, et al., Gene, 157:261-4, 1995; WO 9746705 and WO 9515373.

Correlation of aberrant DNA methylation with cancer. Aberrant DNA methylation within CpG ‘islands’ is characterized by hyper- or hypomethylation of CpG dinucleotide sequences leading to abrogation or overexpression of a broad spectrum of genes, and is among the earliest and most common alterations found in, and correlated with human malignancies. Additionally, abnormal methylation has been shown to occur in CpG-rich regulatory elements in intronic and coding parts of genes for certain tumors. In colon cancer, aberrant DNA methylation constitutes one of the most prominent alterations and inactivates many tumor suppressor genes such as p14ARF, p16INK4a, THBS1, MINT2, and MINT31 and DNA mismatch repair genes such as hMLH1.

In contrast to the specific hypermethylation of tumor suppressor genes, an overall hypomethylation of DNA can be observed in tumor cells. This decrease in global methylation can be detected early, far before the development of frank tumor formation. A correlation between hypomethylation and increased gene expression has been determined for many oncogenes.

Colorectal cancer. DNA methylation errors have been suggested to play two distinct roles in the molecular evolution of colorectal cancer. In normal colonic mucosa cells, methylation errors accumulate as a function of age or as time-dependent events predisposing these cells to neoplastic transformation. For example, hypermethylation of several loci has been been shown to be already present in adenomas, particularly in the tubulovillous and villous subtype. At later stages, increased DNA methylation of CpG islands plays an important role in a subset of tumors affected by the so-called “CpG island methylator phenotype” (CIMP). Most CIMP-positive tumors, which constitute about 15% of all sporadic colorectal cancers, are characterized by microsatellite instability (MIN) due to hypermethylation of the hMLH1 promoter and other DNA mismatch repair genes. By contrast, CIMP-negative colon cancers evolve along a more classic genetic instability pathway (CIN), with a high frequency of p53 mutations and chromosomal changes.

These colon cancer subtypes, in addition to displaying varying frequencies of molecular alteration (e.g., MIN vs CIN), can be subclassified into two significantly different clinical classes. Almost all MIN tumors originate in the proximal colon (ascending and transversum), whereas 70% of CIN tumors are located in the distal colon and rectum. This spatial distinction has been attributed to the varying prevalence of different carcinogens in different sections of the colon. Methylating carcinogens, which constitute the prevailing carcinogen in the proximal colon are implicated in the pathogenesis of MIN cancers, whereas CIN tumors appear to be frequently caused by adduct-forming carcinogens that occur more frequently in distal parts of the colon and rectum. Moreover, MIN tumors have a better prognosis than do tumors with a CIN phenotype and respond better to adjuvant chemotherapy.

Breast cancer. Breast cancer is defined as the uncontrolled proliferation of cells within breasts tissues. Breasts are comprised of 15 to 20 lobes joined together by ducts. Cancer arises most commonly in the duct, but is also found in the lobes with the rarest type of cancer, termed inflammatory breast cancer.

Breast cancer is currently the second most common type of cancer amongst women. For example, in 2001, over 190,000 new cases of invasive breast cancer and over 47, 000 additional cases of in situ breast cancer were diagnosed in the United States. Incidence and death rates increase with age. For example, during the period from 1994-1998 the incidence of breast cancer among women 20-24 years of age was only 1.5 per 100,000 population. The risk increases to 489.7 per 100,000 population within the 75-79 year age group. Mortality rates have decreased by approximately 5% over the last decade and factors affecting 5-year survival rates include age, stage of cancer, socioeconomic factors and race.

Methods of treatment include the use of surgery, radiation therapy, chemotherapy and hormone therapy, which are also used as adjunct therapies to surgery. The first step of any treatment is the assessment of the patient's condition, comparative to defined classifications of the disease. Typically, breast cancers are staged according to size, location and occurrence of metastasis. However, the value of such a system is inherently dependant upon the quality of the classification and, in contrast to the detection of some other common cancers such as cervical and dermal, there are inherent difficulties in classifying and detecting breast cancers.

Additional predictors (e.g., histological analysis, estrogen receptor markers, etc.) currently used in, or to supplement the assessment of breast tumors often fail to allow for correct prediction or classification of tumor development and behavior. Consequently, patient response to treatment is often not accurately predictable, and prediction of overall outcome is problematic.

The continued development of breast cancer analysis techniques is currently focused upon the investigation of molecular biological markers. The development of molecular biological markers as an alternative to traditional histopathological analysis has focused on the analysis of single-nucleotide polymorphisms (SNPs) and single genes, such as BRCA1 and BRCA 2. Furthermore, gene amplification and loss of heterozygosity have been used, in addition to such oncogene mutations, to assess invasive breast cancer. More recently, the use of microarray technology and gene expression profiling has allowed the concurrent analysis of multiple genes as well as the genetic expression profiling by analysis of RNA and proteins (Friend et. al., Nature 415:530-536, 2002; using gene expression profiling to predict the outcome of treatment in breast cancer patients).

However, hereditary breast cancers account for only 5% to 10% of cases, and epigenetic mechanisms, as well as environmental factors influence the development of breast cancers.

The calcitonin gene. The short (“P”) arm of chromosome 11 is the location of several tumor suppressor genes, including the calcitonin gene. Carcinogenesis in multiple types of cancers has been associated with hypomethylation of this region.

The alpha-calcitonin gene encodes a small family of peptides comprising calcitonin, katacalcin, and calcitonin gene-related peptide (CGRP). Calcitonin and katacalcin are produced from one precursor, and CGRP from another. Calcitonin and katacalcin are primarily produced in/from the thyroid, while CGRP is present in both the thyroid and the central nervous system. Calcitonin is involved with skeletal integrity, and the secretion of calcitonin is, at least in part, oestrogen dependent. Thus, it is likely that a postmenopausal decline in calcitonin secretion is a factor in the development of postmenopausal osteoporosis, and calcitonin may prove useful in the prevention and perhaps the treatment of this condition.

Investigation of the Calcitonin gene has revealed that hypermethylation of the promoter region of the gene is present in neoplastic cells of several cancer types including acute leukaemia's. Examples of research carried out using restriction enzyme based methods on the calcitonin gene promoter and/or first exon include the following: colon cancer (Hiltunen et al., Br J Cancer, 76:1124-30, 1997; Silverman et al., Cancer Res., 49:3468-7, 1989); leukaemia (Roman et al., Br J Haematol., 113:329-3, 2001); and breast cancer (Hakkarainen et al., Int J Cancer, 69:471-4, 1996); myelodysplastic syndrome (Dhodapkar et al., LeukRes., 19:719-26, 1995).

However, while implicating calcitonin epigenetic factors in multiple types of cancers, these studies are significantly limited in scope. Specifically, such investigations were primarily carried out using methylation-sensitive restriction enzyme-based methods, and have thus identified only a limited number of specific CpG hypermethylation events, being located only within specific promoter and first exon regions of the calcitonin gene.

More recently, bisulphite-based methods have allowed a slightly broader analysis of methylation patterns within the calcitonin gene (e.g., Silverman et al., Cancer Res., 49:346873, 1989; Hiltunen et al., Br J Cancer, 76:1124-30, 1997). Here again, however, these investigations have concentrated upon the analysis of particular CpG dinucleotides within the calcitonin first exon and promoter regions.

Significantly, said prior art methods and findings do not validate the potential diagnostic and/or prognostic utility of determination of methylation status at other CpG positions located elsewhere within, or in the proximity of the calicitonin gene, particularly where such limited prior art-analyzed CpG positions are not part of CpG islands.

As mentioned herein above in relation to a number of genes involved with cancer, it has been shown that methylation of the correlating promoter region is involved in the regulation of gene expression. For example, in prostate carcinoma patients the promoter of the gene GSTP1 (glutathionyltransferase P1) is hypermethylated, resulting in silencing of GSTP1 expression. To date, however, CpG dinucleotides and/or CpG islands lying further upstream of the Calcitonin gene have not been associated with the development of cancers.

It will be appreciated by those skilled in the art that there exists a continuing need to improve existing methods of early detection, classification and treatment of cancer and proliferative disorders including, inter alia, leukaemia, breast cancer, colon cancer, and myelodysplastic syndrome. There is also an urgent need in the art to discover and utilize novel predictive associations with such cancers and proliferative disorders, and particularly predictive associations relating to epigenetic events within, and in the proximity of the calcitonin gene.

Additional relevant prior art methods. An overview of the Prior Art in oligomer array manufacturing can be gathered from a special edition of Nature Genetics (Nature Genetics Supplement, Volume 21, January 1999, and from the literature cited therein).

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut & Beck, Current Innovations and Future Trends, 1: 147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which make the detection of unmodified substrates considerably more difficult.

### SUMMARY OF THE INVENTION

The present invention provides novel methods for the analysis of cell proliferative disorders involving analysis of a novel CpG island that was heretofore not associated with the development of cancer. Furthermore, the invention discloses genomic and chemically modified nucleic acid sequences, as well as oligonucleotides and/or PNA-oligomers for analysis of cytosine methylation patterns within said region.

The present invention is in part based on the discovery that genetic and epigenetic parameters, in particular, the cytosine methylation patterns, of a novel CpG-rich region of the genome, upstream of the calcitonin gene, are particularly useful for the diagnosis, prognosis, management and/or therapy of cancer and other cell proliferative disorders.

### BRIEF DESCRIPTION OF THE DRAWINGS

FIG. 1 shows the analysis of bisulphate-treated DNA using the MethylLight™ assay, performed according to EXAMPLE 1, herein below. The Y-axis shows the percentage of methylation at the CpG positions covered by the probes. The dark grey bar (“A” in the legend) corresponds to tumor samples, whereas the white bar (“B”) corresponds to normal control tissue. The tumor samples are hypermethylated relative to normal control tissue.

FIG. 2 shows the amplification of bisulphate-treated DNA according to EXAMPLE 2, herein below. The lower trace (“B”) shows the amplification of DNA from normal colon tissue, while the upper trace (“A”) shows the amplification of DNA from tumor tissue. The X-axis shows the cycle number of the amplification whereas the Y-axis shows the amount of amplificate detected.

FIG. 3 shows the analysis of bisulphate-treated DNA using the combined HeavyMethyl MethylLight assay according to EXAMPLE 2, herein below. The X-axis shows the percentage of methylation at the CpG positions covered by the probes. The dark grey bar represents tumor samples, whereas the white bar represents normal control tissue.

FIG. 4 shows the level of methylation in breast tumor and healthy tissues as assessed according to EXAMPLE 2, herein below (by means of the Heavy Methyl assay). The Y-axis shows the degree of methylation within the region of the Calcitonin gene investigated. Tumor samples are represented by black diamonds, and normal breast tissue samples by white squares. As can be seen from the results, a significantly higher degree of methylation (hypermethylation) was observed in tumor samples relative to normal tissue samples.

FIG. 5 shows a methylation analysis of bisulphate-treated DNA from breast tumour and normal control tissue using the MethylLight™ assay, (according to EXAMPLE 4, herein below), and the combined HeavyMethyl MethylLight™ assay (according to EXAMPLE 5, herein below). The Y-axis shows the percentage of methylation at the CpG positions covered by the probes. The black bars correspond to tumor samples, whereas the white bars correspond to normal control tissue. The bar charts on the left hand side of the X-axis show the percentage methylation measured using the combined (HeavyMethyl™) assay while the bar charts on the right show the analysis carried out by means of the MethylLight™ assay. Analysis by means of both assays shows that the breast tumor samples are significantly hypermethylated relative to normal control tissue.

### DETAILED DESCRIPTION OF THE INVENTION

Definitions:

The term “Observed/Expected Ratio” (“O/E Ratio”) refers to the frequency of CpG dinucleotides within a particular DNA sequence, and corresponds to the [number of CpG sites/(number of C bases x number of G bases)] x band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length.

The term “methylation state” or “methylation status” refers to the presence or absence of 5-methylcytosine (“5-mCyt”) at one or a plurality of CpG dinucleotides within a DNA sequence. Methylation states at one or more particular palindromic CpG methylation sites (each having two CpG CpG dinucleotide sequences) within a DNA sequence include “unmethylated,” “fully-methylated” and “hemi-methylated.”

The term “hemi-methylation” or “hemimethylation” refers to the methylation state of a palindromic CpG methylation site, where only a single cytosine in one of the two CpG dinucleotide sequences of the palindromic CpG methylation site is methylated (e.g, 5′-CC^{M}GG-3′ (top strand): 3′-GGCC-5′ (bottom strand)).

The term “hypermethylation” refers to the methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both ‘DNA microarrays,’ and ‘DNA chip(s),’ as recognized in the art, encompasses all art-recognized solid supports, and encompasses all methods for affixing nucleic acid molecules thereto or synthesis of nucleic acids thereon.

The term “hybridization” is to be understood as a bond of an oligonucleotide to a completely complementary sequence along the lines of the Watson-Crick base pairings in the sample DNA, forming a duplex structure.

“Stringent hybridization conditions” are those conditions in which a hybridization is carried out at 60° C. in 2.5×SSC buffer, followed by several washing steps at 37° C. in a low buffer concentration, and remains stable.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlates with the DNA methylation.

CpG Dinucleotide Sequences within a CpG-Rich Region (CpG-Island) Upstream of the Calcitonin Gene were Determined to be useful for the Diagnosis, Prognosis, Management and/or Therapy of Cancer and other Cell-Proliferative Disorders:

The present invention is based upon the identification of a CpG-rich region within the chromosomal region of the calcitonin gene family, and lying upstream (5′) of the calcitonin gene (Genbank accession number X15943). Heretofore, said CpG-rich island had not been associated with tumorigenesis and/or other proliferative disorders; previously published research concerning methylation analysis within the calcitonin gene being limited in scope to the associated promoter and first exon regions. The herein disclosed CpG-rich region lies approximately 1000 base pairs (1 Kb) upstream of the transcription start site of the calcitonin gene. Previsously, cancer-associated methylation patterns have only been associated with particular CpG dinucleotide sequences occurring closer to the vicinity of the transcription start site of said gene. An objective of the present invention is to provide improved methods for the diagnosis, prognosis, management and/or therapy of cell proliferative disorders by analysis of said novel CpG island.

The present invention provides novel methods for the analysis of cell proliferative disorders involving analysis of a novel CpG island that was heretofore not associated with the development of cancer. Furthermore, the invention discloses genomic and chemically modified nucleic acid sequences, as well as oligonucleotides and/or PNA-oligomers for analysis of cytosine methylation patterns within said region.

The present invention is in part based on the discovery that genetic and epigenetic parameters, in particular, the cytosine methylation patterns, of a novel CpG-rich region of the genome, upstream of the calcitonin gene, are particularly useful for the diagnosis, prognosis, management and/or therapy of cancer and other cell proliferative disorders.

An objective of the invention comprises analysis of the methylation state of the CpG dinucleotides within the genomic sequence according to SEQ ID NO:1 and sequences complementary thereto. SEQ ID NO:1 corresponds to a fragment of the CpG-rich region upstream of the calcitonin gene, wherein said fragment contains CpG dinucleotides exhibiting one or more disease-specific CpG methylation patterns. The methylation pattern of said fragment of the gene Calcitonin has heretofore not been analysed with regard to cancer and/or other cell proliferative disorders.

In a preferred embodiment of the method, the objective comprises analysis of a chemically modified nucleic acid comprising a sequence of at least 18 bases in length, according to one of SEQ ID NO:2 to SEQ ID NO:5 and sequences complementary thereto. SEQ ID NO:2 through SEQ ID NO:5 provide chemically modified versions of the nucleic acid according to SEQ ID NO:1, wherein the chemical modification of said sequence results in the synthesis of a nucleic acid having a sequence that is unique and distinct from SEQ ID NO:1. Heretofore, the nucleic acid molecules according to SEQ ID NO:1 to SEQ ID NO:5 could not and were connected with the ascertainment of genetic and epigenetic parameters relevant to the analysis of cancer and/or other cell proliferative disorders.

In an alternative preferred embodiment, such analysis comprises the use of an oligonucleotide or oligomer for detecting the cytosine methylation state within genomic or pretreated (chemically modified) DNA, according to SEQ ID NO:1 to SEQ ID NO:5. Said oligonucleotide or oligomer containing at least one base sequence having a length of at least nine (9) nucleotides which hybridizes to a pretreated nucleic acid sequence according to SEQ ID NO:2 to SEQ ID NO:5 and/or sequences complementary thereto, or to a genomic sequence comprising SEQ ID NO:1 and/or sequences complementary thereto.

The oligonucleotides or oligomers according to the present invention constitute novel and effective tools useful to ascertain genetic and epigenetic parameters of the novel CpG rich island disclosed herein. The base sequence of said oligonucleotides or oligomers preferably contain at least one CpG, TpG or CpA dinucleotide. The probes may also exist in the form of a PNA (peptide nucleic acid) which has particularly preferred pairing properties. Particularly preferred oligonucleotides or oligomers according to the present invention are those in which the cytosine of the CpG dinucleotide is within the middle third of the oligonucleotide; that is, where the oligonucleotide is, for example, 13 bases in length, the CG, TG or CA dinucleotide is positioned within the fifth to ninth nucleotide from the 5′-end.

The oligonucleotides or oligomers according to particular embodiments of the present invention are typically used in ‘sets,’ which contain at least one oligomer for analysis of each of the CpG dinucleotides of genomic sequence SEQ ID NO:1 and sequences complementary thereto, or to the corresponding CpG, TpG or CpA dinucleotide within a sequence of the pretreated nucleic acids according to SEQ ID NO:2 to SEQ ID NO:5 and sequences complementary thereto.

In a preferred embodiment, an oligonucleotide set contains at least one oligomer for each of the CpG dinucleotides within the gene Calcitonin in both the pretreated and genomic versions of said gene sequence according to SEQ ID NO2 through SEQ ID NO:5 and SEQ ID NO:1, respectively. However, it is anticipated that for economic or other factors it may be preferable to analyze a limited selection of the CpG dinucleotides within said sequences and the content of the set of oligonucleotides is altered accordingly.

Therefore, in particular embodiments, the present invention provides a set of at least three (3) (oligonucleotides and/or PNA-oligomers) useful for detecting the cytosine methylation state in pretreated genomic DNA (SEQ ID NO:2 to SEQ ID NO:5 and sequences complementary thereto) and genomic DNA (SEQ ID NO:1 and sequences complementary thereto). These probes enable diagnosis, prognosis, and/or therapy of genetic and epigenetic parameters of cell proliferative disorders. The set of oligomers may also be used for detecting single nucleotide polymorphisms (SNPs) in pretreated genomic DNA (SEQ ID NO:2 to SEQ ID NO:5, and sequences complementary thereto) and genomic DNA (SEQ ID NO:1, and sequences complementary thereto).

In further embodiments, the present invention provides a set of at least two (2) oligonucleotides that are used as ‘primer’ oligonucleotides for amplifying DNA sequences of one of SEQ ID NO:1 to SEQ ID NO:5 and sequences complementary thereto, or segments thereof.

In preferred embodiments, at least one, and more preferably all members of a set of oligonucleotides is bound to a solid phase.

In particular embodiments, it is preferred that an arrangement of different oligonucleotides and/or PNA-oligomers (a so-called “array”), made according to the present invention, is present in a manner that it is likewise bound to a solid phase. Such an array of different oligonucleotide- and/or PNA-oligomer sequences can be characterized, for example, in that it is arranged on the solid phase in the form of a rectangular or hexagonal lattice. The solid-phase surface is preferably composed of silicon, glass, polystyrene, aluminum, steel, iron, copper, nickel, silver, or gold. However, nitrocellulose as well as plastics such as nylon, which can exist in the form of pellets or also as resinmatrices, may also be used.

Therefore, in further embodiments, the present invention provides a method for manufacturing an array fixed to a carrier material for analysis in connection with cell proliferative disorders, in which method at least one oligomer according to the present invention is coupled to a solid phase. Methods for manufacturing such arrays are known and described in, for example, U.S. Pat. No. 5,744,305 by means of solid-phase chemistry and photo labile protecting groups.

The present invention further provides a DNA chip for the analysis of cell proliferative disorders. DNA chips are known and described in, for example, U.S. Pat. No. 5,837,832.

Additionally, a subject matter of the present invention comprises a ‘kit’ which may be composed, for example, of a bisulfite-containing reagent, a set of primer oligonucleotides containing at least two oligonucleotides whose sequences in each case corresponds b or are complementary to an 18-base long segment of the nucleic acid sequences of SEQ ID NO:1 to SEQ ID NO:5 and sequences complementary thereto, oligonucleotides and/or PNA-oligomers, as well as instructions for carrying out and evaluating the described method. However, a kit of the present invention can also contain only part of the aforementioned components.

The present invention further provides a method for ascertaining genetic and/or epigenetic parameters of the calcitonin gene within a subject by analyzing cytosine methylation and single nucleotide polymorphisms. Said method comprising contacting a nucleic acid comprising one or more sequences, from the group consisting of SEQ ID NO:1 through SEQ ID NO:5, in a biological sample obtained from said subject with at least one reagent or a series of reagents, wherein said reagent or series of reagents, distinguishes between methylated and non methylated CpG dinucleotides within the target nucleic acid.

Preferably, said method comprises the following steps: In the first step, obtaining a sample of the tissue to be analysed. The source may be any suitable source, such as cells or cell components, cell lines, biopsies, blood, sputum, stool, urine, cerebrospinal fluid, tissue embedded in paraffin such as tissue from eyes, intestine, kidney, brain, heart, prostate, lung, colon, breast or liver, histologic object slides, or combinations thereof.

In the second step, DNA is isolated from the sample. Extraction may be by means that are standard to one skilled in the art, these include the use of detergent lysates, sonification and vortexing with glass beads. Once the nucleic acids have been extracted the genomic double stranded DNA is used in the analysis.

In the third step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ herein.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In the fourth step of the method, fragments of the pretreated DNA are amplified, using sets of primer oligonucleotides according to the present invention, and a preferably heat-stable polymerase. Because of statistical and practical considerations, preferably more than ten different fragments having a length of from about 100 to about 2,000 base pairs are amplified. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary or identical to an at least 18-base-pair long segment of the base sequences of SEQ ID NO:1 to SEQ ID NO:5 and sequences complementary thereto.

In an alternate embodiment of the method, the methylation status of preselected CpG positions within the nucleic acid sequences comprising SEQ ID NO:2 to SEQ ID NO:5 may be detected by use of methylation-specific primer oligonucleotides. This technique (MSP) has been described in U.S. Pat. No. 6,265,171 to Herman. The use of methylation status specific primers for the amplification of bisulphite treated DNA allows the differentiation between methylated and unmethylated nucleic acids. MSP primers pairs contain at least one primer which hybridizes to a bisulphite treated CpG dinucleotide. Therefore the sequence of said primers comprises at least one CG, TG or CA dinucleotide. MSP primers specific for non-methylated DNA contain a “T’ at the 3′ position of the C position in the CpG. Preferably, therefore, the base sequence of said primers is required to comprise a sequence having a length of at least 9 nucleotides which hybridizes to a pretreated nucleic acid sequence according to SEQ ID NO:2 to SEQ ID NO:5 and sequences complementary thereto, wherein the base sequence of said oligomers comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

In the fifth step of the method, the amplificates obtained during the fourth step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesised in step four are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; and said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In a preferred embodiment said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to Seq. ID No. 1, and the equivalent positions within SEQ ID NOs:2 to 5. Said oligonucleotides may also be present in the form of peptide nucleic acids. The non-hybridized amplificates are then removed.

In the final step of the method, the hybridized amplificates are detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridised to the bisulphite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulphite treated DNA, and moreover for methylation analysis within CpG dinucleotides.

A further suitable method for the use of probe oligonucleotides for the assessment of methylation by analysis of bisulphite treated nucleic acids comprises the use of blocker oligonucleotides. The use of such blocker oligonucleotides has been described by Yu et al., BioTechniques 23:714-720, 1997. Blocking probe oligonucleotides are hybridized to the bisulphate-treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulphite treated nucleic acid in a methylation status specific manner. For example, for detection of methylated nucleic acids within a population of unmethylated nucleic acids, suppression of the amplification of nucleic acids which are unmethylated at the position in question would be carried out by the use (f blocking probes comprising a ‘CpG’ at the position in question, as opposed to a ‘CpA.’

In a further preferred embodiment of the method, the fifth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fifth step of the method comprises sequencing and subsequent sequence analysis of the amplificate generated in the third step of the method (Sanger F., et al., PNAS USA 74:5463-5467, 1977).

Additional embodiments of the invention provide a method for the analysis of the methylation status of genomic DNA according to the invention (SEQ ID NO:1) without the need for pretreatment.

In the first step of such additional embodiments, the genomic DNA sample is isolated from tissue or cellular sources. Preferably, such sources include cell lines, histological slides, body fluids, or tissue embedded in paraffin. Extraction may be by means that are standard to one skilled in the art, including but not limited to the use of detergent lysates, sonification and vortexing with glass beads. Once the nucleic acids have been extracted the genomic double-stranded DNA is used in the analysis.

In a preferred embodiment the DNA may be cleaved prior to the treatment, this may be any means standard in the state of the art, in particular with methylation-sensitive restriction endonucleases. In the second step, the DNA is then digested with one or more methylation sensitive restriction enzymes. The digestion is carried out such that hydrolysis of the DNA at the restriction site is informative of the methylation status of a specific CpG dinucleotide.

In the third step, which is optional but a preferred embodiment, the restriction fragments are amplified. This is preferably carried out using a polymerase chain reaction.

In the final step the amplificates are detected. The detection may be by any means standard in the art, for example, but not limited to, gel electrophoresis analysis, hybridization analysis, incorporation of detectable tags within the PCR products, DNA array analysis, MALDI or ESI analysis. Suitable labels for use in the detection of the digested nucleic acid fragments include fluorophore labels, radionuclides and mass labels as described above.

The oligomers according to the present invention, or arrays thereof, as well as a kit according to the present invention are useful for the diagnosis and/or therapy of cancer and/or other cell proliferative disorders. According to the present invention, the method is preferably used for the diagnosis and/or therapy of cell proliferative disorders by analysis of important genetic and/or epigenetic parameters within the novel CpG-rich region located upstream (5′) of the calcitonin gene.

The methods according to the present invention are used, for example, for the diagnosis and/or therapy of cell proliferative disorders.

The nucleic acids according to the present invention SEQ ID NO:1 to SEQ ID NO:5, and sequences complementary thereto can be used for the diagnosis and/or therapy of genetic and/or epigenetic parameters associated with the gene Calcitonin.

The present invention moreover relates to a method for manufacturing a diagnostic agent and/or therapeutic agent for the diagnosis and/or therapy of diseases associated with the calcitonin gene, comprising analyzing methylation patterns of said gene, the diagnostic agent and/or therapeutic agent being characterized in that at least one nucleic acid according to the present invention is used for manufacturing it, possibly together with suitable additives and ancillary agents.

A further subject matter of the present invention relates to a diagnostic agent and/or therapeutic agent for diseases associated with the calcitonin gene, comprising analyzing methylation patterns of said gene, the diagnostic agent and/or therapeutic agent containing at least one nucleic acid according to the present invention, possibly together with suitable additives and ancillary agents.

The present invention moreover relates to the diagnosis and/or prognosis of events which are disadvantageous to patients or individuals in which important genetic and/or epigenetic parameters within the gene Calcitonin may be used as markers. Said parameters obtained by means of the present invention may be compared to another set of genetic and/or epigenetic parameters, the differences serving as the basis for a diagnosis and/or prognosis of events which are disadvantageous to patients or individuals.

Moreover, a subject matter of the present invention is a kit comprising, for example, a bisulfite-containing reagent, a set of primer oligonucleotides containing at least two oligonucleotides whose sequences in each case correspond or are complementary to a 18-base long segment of the base sequences specified in the appendix (SEQ ID NO:1 through SEQ ID NO:5), oligonucleotides and/or PNA-oligomers as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethylLight™, Heavy Methyl™, and nucleic acid sequencing. However, a kit along the lines of the present invention can also contain only part of the aforementioned components.

### EXAMPLES

In the following examples, methylation status of the CpG island disclosed under SEQ ID NO:1 was analyzed using two alternative methods. In the first example, a real time PCR was carried out upon bisulphate-treated DNA using fluorescent labeled probes in a real time PCR assay covering CpG positions of interest (a variant of the Taqman assay known as the MethyLight™ assay). In the second experiment methylation status of the same region was analyzed by bisulphite treatment. This was followed by analysis of the treated nucleic acids using a MethylLight™ assay combined with the methylation specific blocking probes covering CpG positions (HeavyMethyl™ assay).

### Example 1

### Methylation within Colon Cancer Tissue was Analyzed using a MethyLight™ Assay

DNA was extracted from 34 colon adenocarcinoma samples and 42 colon normal adjacent tissues using a Qiagen™ extraction kit. The DNA from each sample was treated using a bisulfite solution (hydrogen sulfite, disulfite) according to the agarose-bead method (Olek et al 1996). The treatment is such that all non methylated cytosines within the sample are converted to thymidine. Conversely, 5-methylated cytosines within the sample remain unmodified.

The methylation status was determined with a MethyLight™ assay designed for the CpG island of interest and a control fragment from the beta actin gene (Eads et al., 2001). The CpG island assay covers CpG sites in both the primers and the Taqman™ style probe, while the control gene does not. The control gene is used as a measure of total DNA concentration, and the CpG island assay (methylation assay) determines the methylation levels at that site.

Methods. The calcitonin gene CpG island assay was performed using the following primers and probes:

| | | |
|---|---|---|
| Primer: | AGGTTATCGTCGTGCGAGTGT; | (SEQ ID NO:6) |
| Primer: | TCACTCAAACGTATCCCAAACCTA; | (SEQ ID NO:7) |
| and | | |
| Probe: | CGAATCTCTCGAACGATCGCATCCA. | (SEQ ID NO:8) |

The corresponding control assay was performed using the following primers and probes

| | |
|---|---|
| Primer: | |
| TGGTGATGGAGGAGGTTTAGTAAGT; | (SEQ ID NO:9) |
| Primer: | |
| AACCAATAAAACCTACTCCTCCCTTAA; | (SEQ ID NO:10) |
| and | |
| Probe: | |
| ACCACCACCCAACACACAATAACAAACACA. | (SEQ ID NO:11) |

The reactions were run in triplicate on each DNA sample with the following assay conditions:

Reaction solution: (900 nM primers; 300 nM probe; 3.5 mM magnesium chloride; 1 unit of taq polymerase; 200 μM dNTPs; 7 μl of DNA, in a final reaction volume of 20 μl);

Cycling conditions: (95° C. for 10 minutes; 95° C. for 15 seconds; 67° C. for 1 minute (3 cycles)); (95° C. for 15 seconds, 64° C. for 1 minute (3 cycles)); (95° C. for 15 seconds, 62° C. for 1 minute (3 cycles)); and (95° C. for 15 seconds, 60° C. for 1 minute (40 cycles)).

The data was analyzed using a PMR calculation previously described in the literature (Eads et al 2001).

Results. The mean PMR for normal samples was 0.19, with a standard deviation of 0.79. None of the normal samples was greater than 2 standard deviations about the normal mean, while 18 of 34 tumor samples reached this level of methylation. The overall difference in methylation levels between tumor and normal samples is significant in a t-test (p=0.002).

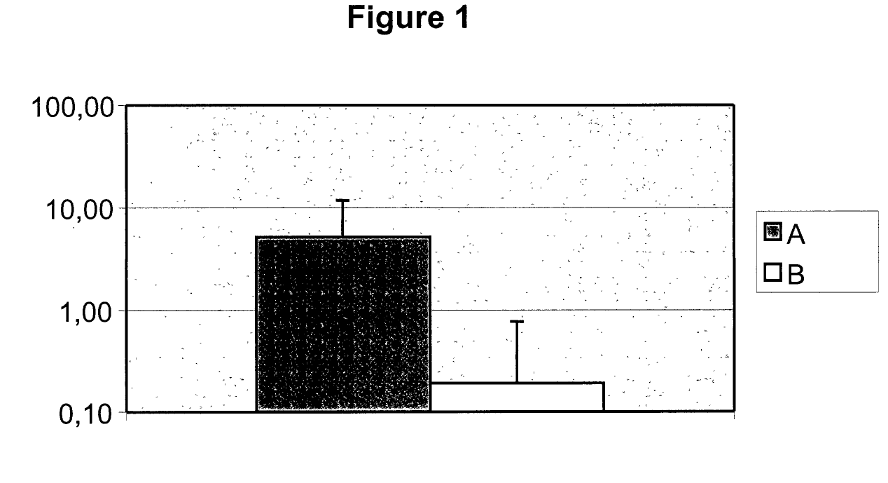

FIG. 1 shows the analysis of bisulphate-treated DNA using the MethylLight™ assay, performed according to this EXAMPLE 1. The Y-axis shows the percentage of methylation at the CpG positions covered by the probes. The dark grey bar (“A” in the legend) corresponds to tumor samples, whereas the white bar (“B”) corresponds to normal control tissue.

Significantly, the tumor samples are substantially hypermethylated relative to normal control tissue.

### Example 2

### Methylation within Colon and Breast Cancer Tissue was Analyzed using a HeavyMethyl MethyLight™ Assay

The colon and normal tissue DNA samples of EXAMPLE 1, above (along with breast cancer and normal breast tissue; see below this EXAMPLE), were also used to analyze methylation of the upstream calcitonin CpG island with a HeavyMethyl MethyLight™ (or HM MethyLight™) assay, also referred to as the HeavyMethyl™ assay. The methylation status was determined with a HM MethyLight™ assay designed for the CpG island of interest and the same control gene assay described above. The CpG island assay covers CpG sites in both the blockers and the Taqman™ style probe, while the control gene does not.

Methods. The CpG island assay (methylation assay) was performed using the following primers and probes:

| | |
|---|---|
| Primer: | |
| GGATGTGAGAGTTGTTGAGGTTA; | (SEQ ID NO:12) |
| Primer: | |
| ACACACCCAAACCCATTACTATCT; | (SEQ ID NO:13) |
| Probe: | |
| ACCTCCGAATCTCTCGAACGATCGC; | (SEQ ID NO:14) |
| and | |
| Blocker: | |
| TGTTGAGGTTATGTGTAATTGGGTGTGA. | (SEQ ID NO:15) |

The reactions were each run in triplicate on each DNA sample with the following assay conditions:

Reaction solution: (300 nM primers; 450 nM probe; 3.5 mM magnesium chloride; 2 units of taq polymerase; 400 μM dNTPs; and 7 μl of DNA, in a final reaction volume of 20 μl;

Cycling conditions: (95° C. for 10 minutes); (95° C. for 15 seconds, 67° C. for 1 minute (3 cycles)); (95° C. for 15 seconds, 64° C. for 1 minute (3 cycles); (95° C. for 15 seconds, 62° C. for 1 minute (3 cycles)); and (95° C. for 15 seconds, 60° C. for 1 minute (40 cycles)).

DNA was extracted from serum samples from 5 of the colon cancer patients with methylation in their tumor samples and 11 healthy controls. The DNA samples were analyzed with the HM MethyLight™ assay and the PMRs were calculated.

Results; colon cancer tissue. The mean PMR for normal samples was 0.13 with a standard deviation of 0.58. None of the normal samples was greater than 2 standard deviations about the normal mean, while 19 of 34 tumor samples reached this level of methylation. The overall difference in methylation levels between tumor and normal samples is significant in a t-test (p=0.0004).

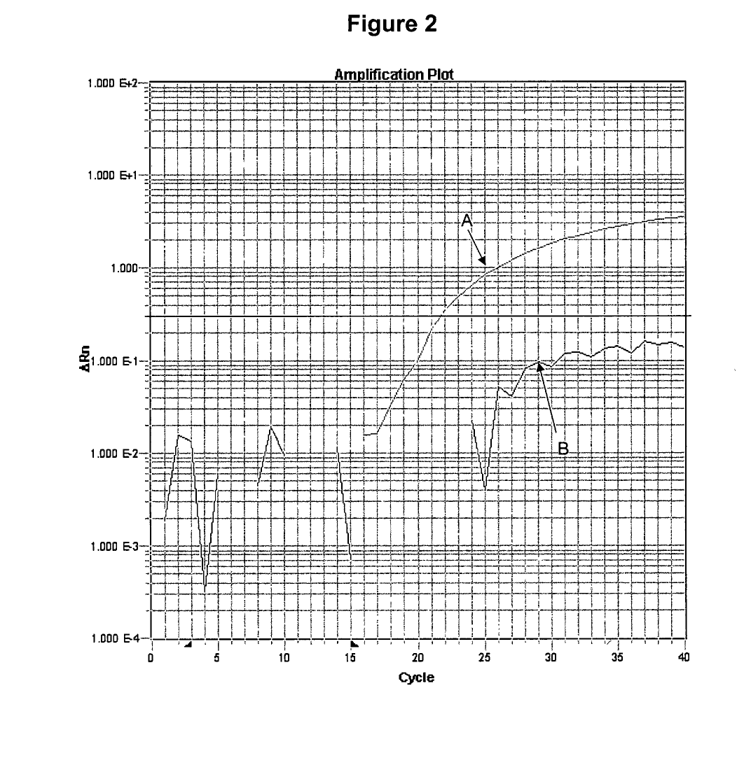

FIG. 2 shows the amplification of bisulphate-treated DNA according to this EXAMPLE 2. The lower trace (“B”) shows the amplification of DNA from normal colon tissue, while the upper trace (“A”) shows the amplification of DNA from tumor tissue. The X-axis shows the cycle number of the amplification, whereas the Y-axis shows the amount of amplificate detected.

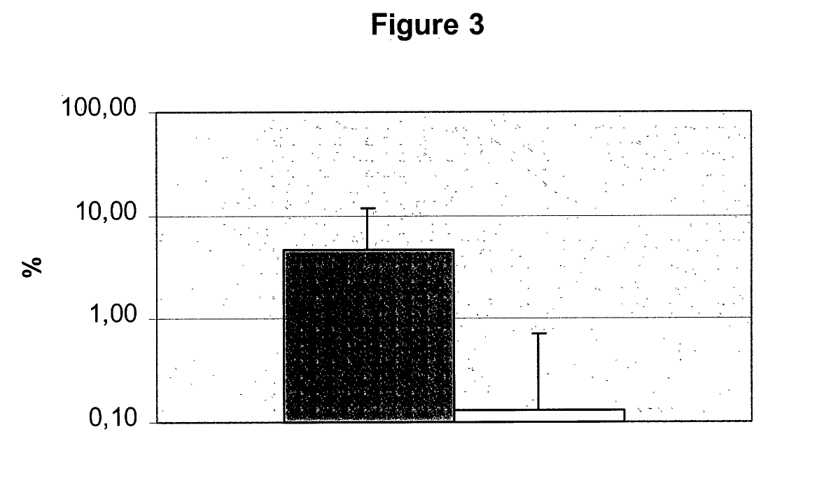

FIG. 3 shows the analysis of bisulphate-treated DNA using the combined HeavyMethyl MethylLight™ assay according to this EXAMPLE 2. The X-axis shows the percentage of methylation at the CpG positions covered by the probes. The dark grey bar represents tumor samples, whereas the white bar represents normal control tissue.

Significantly, all five of the colon cancer patient serum samples had methylation levels at least six standard deviations above the mean of the healthy controls.

Results; breast cancer tissue results. Furthermore, the above described HeavyMethyl™ assay was used to assess methylation differences between eight (8) healthy breast tissue and nine (9) breast tumor samples. All primers, probes, blockers and reaction conditions were identical to those of the colon cancer analyses described above.

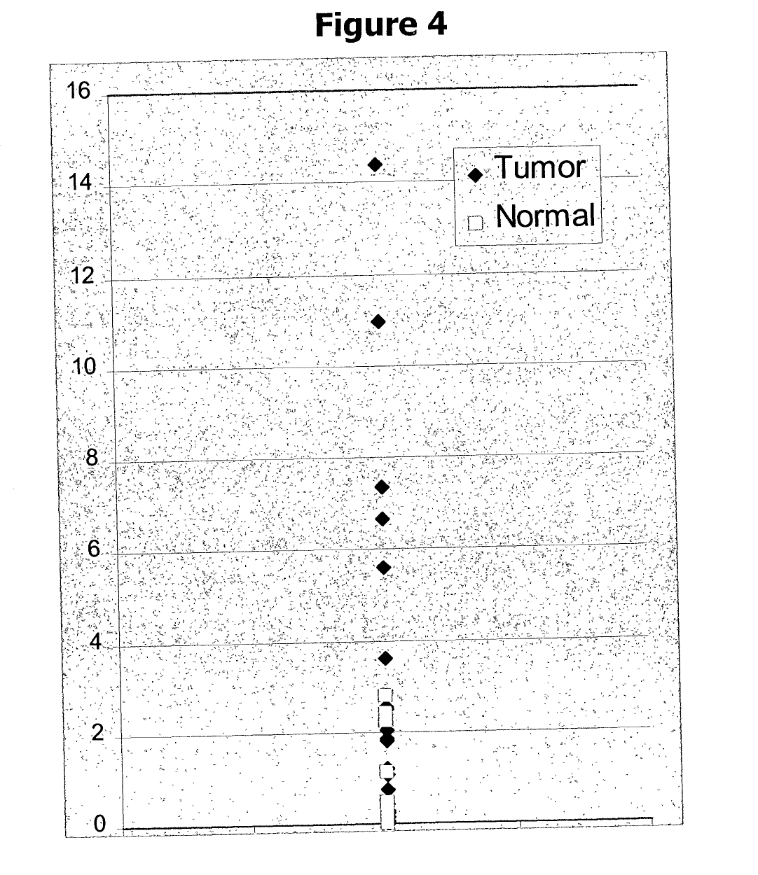

FIG. 4 shows the level of methylation in breast tumor and healthy tissues as assessed according to the methods of this EXAMPLE 2 (by means of the HeavyMethyl™ assay). The Y-axis shows the degree of methylation within the region of the calcitonin gene investigated. Tumor samples are represented by black diamonds, and normal breast tissue samples by white squares. A significantly higher degree of methylation was observed in tumor samples than in healthy tissue samples. The level of significance as measured using a t-test was 0.012. The degree of differences observed between healthy normal and tumor samples using the assay was therefore somewhat higher in colon tissue than in breast tissue (however, see EXAMPLES 4 and 5 herein below, showing results from a larger number of tumor and control samples).

### Example 3

### The Methylation Status of a CpG Dinucleotide Site at Nucleotide Position 576 of SEQ ID NO:1 was Determined using Methylation-Sensitive Restriction Endonuclease Digestion

A fragment of the upstream region of the calcitonin gene (SEQ ID NO:1) was amplified by PCR using the primers CCTTAGTCCCTACCTCTGCT (SEQ ID NO:16) and CTCATTTACACACACCCAAAC (SEQ ID NO:17). The resultant amplificate, 378 bp in length, contained an informative CpG at nucleotide position 165 (corresponding to nucleotide position 576 of SEQ ID NO:1). The amplificate DNA was digested with the methylation-sensitive restriction endonuclease Nar I; recognition motif GGCGCC. Hydrolysis by said endonuclease is blocked by methylation of the CpG at position 165 of the amplificate. The digest was used as a control.

Genomic DNA was isolated from the samples using the DNA Wizzard™ DNA isolation kit (Promega). Each sample was digested using Nar I according to manufacturer's recommendations (New England Biolabs).

About 10 ng of each genomic digest was then amplified using PCR primers CCTTAGTCCCTACCTCTGCT (SEQ ID NO:16) and CTCATTTACACACACCCAAAC (SEQ ID NO:17). The PCR reactions were performed using a thermocycler (Eppendorf GmbH) using 10 ng of DNA, 6 pmole of each primer, 200 μM of each dNTP, 1.5 mM MgCl₂ and 1 Unit of Hotstart™Taq (Qiagen AG). The other conditions were as recommended by the Taq polymerase manufacturer.

Using the above mentioned primers, gene fragments were amplified by PCR performing a first denaturation step for 14 min at 96° C., followed by 30-45 cycles (step 2: 60 sec at 96° C., step 3: 45 sec at 52° C., step 4: 75 sec at 72° C.) and a subsequent final elongation of 10 min at 72° C. The presence of PCR products was analyzed by agarose gel electrophoresis.

PCR products were detectable, with Nar I-hydrolyzed DNA isolated wherein the tissue in question (breast or colon) contained up-methylated DNA, when step 2 to step 4 of the cycle program were repeated 34, 37, 39, 42 and 45-fold. In contrast, PCR products were only detectable with Nar I-hydrolyzed DNA isolated from down-methylated tissue (breast or colon) when steps 2 to step 4 of the cycle program were repeated 42- and 45-fold.

### Example 4

### Methylation was Analyzed in Breast Cancer Tissue using a MethyLight™ Assay

DNA was extracted from 21 breast carcinoma samples and 17 normal breast tissues using a Qiagen extraction kit. The DNA from each sample was treated using a bisulfite solution (hydrogen sulfite, disulfite) according to the agarose-bead method (Olek et al 1996). The treatment is such that all non-methylated cytosines within the sample are converted to thymidine. Conversely, 5-methylated cytosines within the sample remain unmodified.

The methylation status was determined with a MethyLight™ assay designed for the CpG island of interest and a control fragment from the beta actin gene (Eads et al., 2001). The CpG island assay covers CpG sites in both the primers and the Taqman™ style probe, while the control gene does not. The control gene is used as a measure of total DNA concentration, and the CpG island assay (methylation assay) determines the methylation levels at that site.

Methods. The calcitonin gene CpG island assay was performed using the following primers and probes:

| | | |
|---|---|---|
| Primer: | AGGTTATCGTCGTGCGAGTGT; | (SEQ ID NO:6) |
| Primer: | TCACTCAAACGTATCCCAAACCTA; | (SEQ ID NO:7) |
| and | | |
| Probe: | CGAATCTCTCGAACGATCGCATCCA. | (SEQ ID NO:8) |

The corresponding control assay was performed using the following primers and probes

| | |
|---|---|
| Primer: | |
| TGGTGATGGAGGAGGTTTAGTAAGT; | (SEQ ID NO:9) |
| Primer: | |
| AACCAATAAAACCTACTCCTCCCTTAA; | (SEQ ID NO:10) |
| and | |
| Probe: | |
| ACCACCACCCAACACACAATAACAAACACA. | (SEQ ID NO:11) |

The reactions were run in triplicate on each DNA sample with the following assay conditions:

Reaction solution: (900 nM primers; 300 nM probe; 3.5 mM Magnesium Chloride; 1 unit of taq polymerase; 200 μM dNTPs; 7 ml of DNA, in a final reaction volume of 20 μl);

Cycling conditions: (95° C. for 10 minutes; 95° C. for 15 seconds; 67° C. for 1 minute (3 cycles)); (95° C. for 15 seconds, 64° C. for 1 minute (3 cycles)); (95° C. for 15 seconds, 62° C. for 1 minute (3 cycles)); and (95° C. for 15 seconds, 60° C. for 1 minute (40 cycles)).

The data was analyzed using a PMR calculation previously described in the literature (Eads et al 2001).

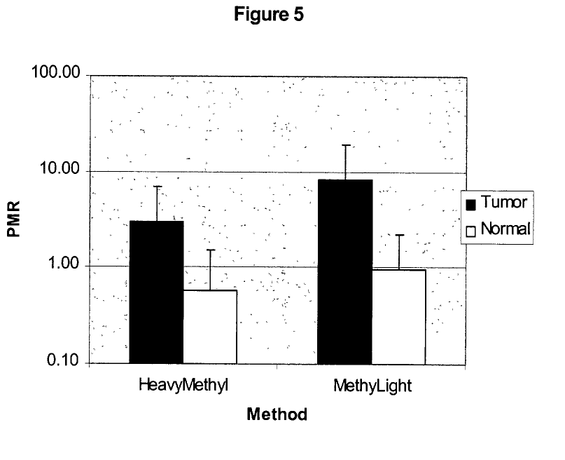

Results. The bar charts on the right-half of FIG. 5 shows the methylation analysis of bisulphate-treated DNA carried out by means of the MethylLight™ assay, performed according to this EXAMPLE 4 (the left-half of this figure shows results from the combined HeavyMethyl MethylLight™ assay according to EXAMPLE 5, herein below). The Y-axis shows the percentage of methylation at the CpG positions covered by the probes. The black bars correspond to tumor samples, whereas the white bars correspond to normal control tissue.

The mean PMR for normal samples was 0.94, with a standard deviation of 1.28. The mean PMR for tumor samples was 8.38, with a standard deviation of 11.18. The overall difference in methylation levels between tumor and normal samples is significant in a t-test (p=0.0065).

Significantly, analysis by means of both assays shows that the tumor samples are significantly hypermethylated relative to normal control tissue.

### Example 5

### Methylation in Breast Cancer Tissue was Analyzed using a HeavyMethyl MethyLight™ Assay

The breast tumour and normal samples of Example 4, above, were also analyzed using the HeavyMethyl MethyLight™ (or HM MethyLight™) assay, also referred to as the HeavyMethyl™ assay. The methylation status was determined with a HM MethyLight™ assay designed for the CpG island of interest and a control gene assay. The CpG island assay covers CpG sites in both the blockers and the Taqman™ style probe, while the control gene does not.

Methods. The calcitonin gene CpG island assay (methylation assay) was performed using the following primers and probes:

| | |
|---|---|
| Primer: | |
| GGATGTGAGAGTTGTTGAGGTTA; | (SEQ ID NO:12) |
| Primer: | |
| ACACACCCAAACCCATTACTATCT; | (SEQ ID NO:13) |
| Probe: | |
| ACCTCCGAATCTCTCGAACGATCGC; | (SEQ ID NO:14) |
| and | |
| Blocker: | |
| TGTTGAGGTTATGTGTAATTGGGTGTGA. | (SEQ ID NO:15) |

The reactions were each run in triplicate on each DNA sample with the following assay conditions:

Reaction solution: (300 nM primers; 450 nM probe; 3.5 mM magnesium chloride; 2 units of taq polymerase; 400 μM dNTPs; and 7 ml of DNA, in a final reaction volume of 20 Cycling conditions: (95° C. for 10 minutes); (95° C. for 15 seconds, 67° C. for 1 minute (3 cycles)); (95° C. for 15 seconds, 64° C. for 1 minute (3 cycles); (95° C. for 15 seconds, 62° C. for 1 minute (3 cycles)); and (95° C. for 15 seconds, 60° C. for 1 minute (40 cycles)).

Results. The bar charts on the left-half of FIG. 5 shows the methylation analysis of bisulphate-treated DNA carried out by means of the combined HeavyMethyl MethylLight™ assay, performed according to this EXAMPLE 5 (the right-half of this figure shows results from the MethylLight™ assay according to EXAMPLE 4, herein above). The Y-axis shows the percentage of methylation at the CpG positions covered by the probes. The black bars correspond to tumor samples, whereas the white bars correspond to normal control tissue.

The mean PMR for normal samples was 0.58, with a standard deviation of 0.94. The mean PMR for tumor samples was 3.01, with a standard deviation of 3.91. The overall difference in methylation levels between tumor and normal samples is significant in a t-test (p=0.0012).

Significantly, analysis by means of both assays shows that the tumor samples are significantly hypermethylated relative to normal control tissue.

We claim:

1. A method for detecting the methylation state of the 5′ upstream region of the calcitonin gene within a subject, said method comprising contacting a nucleic acid comprising one or more sequences from the group comprising SEQ ID NO:1 through SEQ ID NO:5 in a biological sample obtained from said subject with at least one reagent or a series of reagents, wherein said reagent or series of reagents, distinguishes between methylated and non methylated CpG dinucleotides within the target nucleic acid.

2. A method for the analysis of cell proliferative disorders by determination of the methylation state of one or more sequences from the group comprising SEQ ID NO:1 through SEQ ID NO:5 according to claim 1.

3. A method according to claim 2 wherein the method is applied to colon cells or breast cells.

4. A nucleic acid molecule comprising a sequence at least 18 bases in length to according to one of the sequences taken from the group comprising SEQ ID NO:1 to SEQ ID NO:5 and sequences complementary thereto.

5. An oligomer, in particular an oligonucleotide or peptide nucleic acid (PNA) oligomer, said oligomer comprising in each case at least one base sequence having a length of at least 9 nucleotides which hybridizes to or is identical to one of the nucleic acid sequences according to SEQ ID NO:1 through SEQ ID NO: 5.

6. The oligomer as recited in claim 5, wherein the base sequence includes at least one CpG dinucleotide.

7. The oligomer as recited in claim 6, characterized in that the cytosine of the CpG dinucleotide is located approximately in the middle third of the oligomer.

8. A set of oligomers, comprising at least two oligomers according to any of claims 5 to 7.

9. A set of oligomers as recited in claim 8, comprising oligomers for detecting the methylation state of all CpG dinucleotides within SEQ ID NO:1 and sequences complementary thereto.

10. A set of at least two oligonucleotides as recited in one of claims 5 through 9, which can be used as primer oligonucleotides for the amplification of DNA sequences of one of SEQ ID NO:1 to SEQ ID NO:5 and sequences complementary thereto.

11. A set of oligonucleotides as recited in one of claims 8 or 9, characterized in that at least one oligonucleotide is bound to a solid phase.

12. Use of a set of oligonucleotides comprising at least three of the oligomers according to any of claims 5 through 11 for detecting the cytosine methylation state and/or single nucleotide polymorphisms (SNPs) within the sequences taken from the group comprising SEQ ID NO:1 to SEQ ID NO:5 and sequences complementary thereto.

13. A method for manufacturing an arrangement of different oligomers (array) fixed to a carrier material for analyzing diseases associated with the methylation state of the CpG dinucleotides of the gene Calcitonin, wherein at least one oligomer according to any of the claims 5 through 11 is coupled to a solid phase.

14. An arrangement of different oligomers (array) obtainable according to claim 13.

15. An array of different oligonucleotide- and/or PNA-oligomer sequences as recited in claim 14, characterized in that these are arranged on a plane solid phase in the form of a rectangular or hexagonal lattice.

16. The array as recited in any of the claims 14 or 15, characterized in that the solid phase surface is composed of silicon, glass, polystyrene, aluminium, steel, iron, copper, nickel, silver, or gold.

17. A DNA- and/or PNA-array for analyzing diseases associated with the methylation state of the Calcitonin gene, comprising at least one nucleic acid according to one of the preceding claims.

18. A method for determining the methylation state within at least one nucleic acid molecule according to one of claims 1 to 3, comprising:
a) obtaining a biological sample containing genomic DNA;
b) extracting the genomic DNA;
c) converting cytosine bases which are unmethylated at the 5-position within said DNA sample, by chemical treatment, to uracil or another base which is dissimilar to cytosine in terms of hybridization behavior;
d) amplifying fragments of the chemically pretreated genomic DNA using sets of primer oligonucleotides according to one of claims 10 or 11 and a polymerase; and
e) identifying the methylation status of one or more cytosine positions.

19. The method as recited in claim 18, characterized in that Step e) is carried out by means of hybridization of at least one oligonucleotide according to claims 5 through 11.

20. The method as recited in claim 18, characterized in that Step e) is carried out by means of hybridization of at least one oligonucleotide according to claims 5 through 11 and extension of said hybridized oligonucleotide(s) by means of at least one nucleotide base.

21. The method as recited in claim 18, characterized in that Step e) is carried out by means of sequencing.

22. The method as recited in claim 18, characterized in that Step d) is carried out using methylation specific primers.

23. The method as recited in claim 18, characterized in that Step e) is carried out by means of a combination of at least two of the methods described in claims 19 through 22.

24. The method as recited in claim 18, characterized in that the chemical treatment is carried out by means of a solution of a bisulfite, hydrogen sulfite or disulfite.

25. A method for the analysis of methylation within a nucleic acid molecule comprising SEQ ID NO:1 comprising:
a) obtaining a biological sample containing genomic DNA;
b) extracting the genomic DNA;
c) digesting the genomic DNA comprising SEQ ID NO:1 with one or more methylation sensitive restriction enzymes; and
d) detection of the DNA fragments generated in the digest of step c

26. A method according to claim 25, wherein the DNA digest is amplified prior to Step d).

27. The method as recited in one of the claims 18 through 22, characterized in that more than ten different fragments having a length of 100-2000 base pairs are amplified.

28. The method as recited in one of claims 18 through 23, characterized in that the amplification of several DNA segments is carried out in one reaction vessel.

29. The method as recited in one of the claims 18 through 24, characterized in that the polymerase is a heat-resistant DNA polymerase.

30. The method as recited in claim 26, characterized in that the amplification is carried out by means of the polymerase chain reaction (PCR).

31. The method as recited in one of the claims 18 through 24, characterized in that the labels of the amplificates are fluorescence labels.

32. The method as recited in one of the claims 18 through 24, characterized in that the labels of the amplificates are radionuclides.

33. The method as recited in one of the claims 18 through 24, characterized in that the labels of the amplificates are detachable molecule fragments having a typical mass which are detected in a mass spectrometer.

34. The method as recited in one of the claims 18 through 24, characterized in that the amplificates or fragments of the amplificates are detected in the mass spectrometer.

35. The method as recited in one of the claims 28 and/or 29, characterized in that the produced fragments have a single positive or negative net charge for better detectability in the mass spectrometer.

36. The method as recited in one of claims 28 through 30, characterized in that detection is carried out and visualized by means of matrix assisted laser desorption/ionization mass spectrometry (MALDI) or using electron spray mass spectrometry (ESI).

37. The method as recited in one of the claims 18 through 31, characterized in that the genomic DNA is obtained from cells or cellular components which contain DNA, sources of DNA comprising, for example, cell lines, histological slides, biopsies, tissue embedded in paraffin and all possible combinations thereof.

38. A kit comprising a bisulfite (=disulfite, hydrogen sulfite) reagent as well as oligonucleotides and/or PNA-oligomers according to one of the claims 5 through 12.

39. A kit according to claim 38, further comprising standard reagents for performing a methylation assay from the group consisting of MS-SNuPE, MSP, MethylLight™, Heavy Methyl™, nucleic acid sequencing and combinations thereof.

40. The use of a nucleic acid according to claim 4, of an oligonucleotide or PNA-oligomer according to one of the claims 5 through 7, of a kit according to one of claim 38 or 39, of an array according to one of the claims 14 through 17, of a set of oligonucleotides according to one of claims 8 through 12 or a method according to one of claims 1 to 3, 13, and 18 to 37 for the characterization, classification, differentiation, grading, staging, and/or diagnosis of cell proliferative disorders, or the predisposition to cell proliferative disorders.

41. The use of a nucleic acid according to claim 4, of an oligonucleotide or PNA-oligomer according to one of the claims 5 through 7, of a kit according to one of claim 38 or 39, of an array according to one of the claims 14 through 17, of a set of oligonucleotides according to one of claims 8 through 12 or a method according to one of claims 1 to 3, 13, and 18 to 37 for the therapy of cell proliferative disorders.


SEQUENCE LISTING (17 sequences)

SEQ ID NO: 1 - DNA, 965 bp, Homo Sapiens
gatcaattaagggcatcttagaagttaggcgttcccgctgcctcctttgagcacggaggccaccaacccctagggggaagagatgtagcgcgaggcaggggtgtcgtgctaagaaatttcgacgcttctggggactgaggacaaaggtgcggacacgaccccggggtacctggagttccgtgactcgcgccacggacggcacacctaggggctaatttctgctctgcctcaaagaacctcaagctagagtccttgcctccgcccacagccccgggatgccgctgctgcgctcaccgcacaggcagcgcccggaccggctgcagcagatcgcgcgctgcgcgttccaccgggagatggtggagacgctgaaaagcttctttcttgccactctggacgctgtgggcggcaagcgccttagtccctacctctgctgagctgaacgctcaggcacagtggaactgaaacccggttccctacctctgctgagctgaacgctcaggcacagtggaactgaaacccggttctgcgggatgtgagagctgttgaggtcacgcgtaattgggtgtgatggagggcgcctgttcgtgatgtgtgcaggtttgatgcaagcaggtcatcgtcgtgcgagtgtgtggatgcgaccgcccgagagactcggaggcaggcttgggacacgtttgagtgaacacctcaggatactcttctggccagtatctgttttttagtgtctgtgattcagagtgggcacatgttgggagacagtaatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacg

SEQ ID NO: 2 - DNA, 965 bp, Artificial Sequence
  chemically treated genomic DNA (Homo sapiens)
gattaattaagggtattttagaagttaggcgttttcgttgttttttttgagtacggaggttattaatttttagggggaagagatgtagcgcgaggtaggggtgtcgtgttaagaaatttcgacgtttttggggattgaggataaaggtgcggatacgatttcggggtatttggagtttcgtgattcgcgttacggacggtatatttaggggttaatttttgttttgttttaaagaattttaagttagagtttttgttttcgtttatagtttcgggatgtcgttgttgcgtttatcgtataggtagcgttcggatcggttgtagtagatcgcgcgttgcgcgttttatcgggagatggtggagacgttgaaaagttttttttttgttattttggacgttgtgggcggtaagcgttttagtttttatttttgttgagttgaacgtttaggtatagtggaattgaaattcggttttttatttttgttgagttgaacgtttaggtatagtggaattgaaattcggttttgcgggatgtgagagttgttgaggttacgcgtaattgggtgtgatggagggcgtttgttcgtgatgtgtgtaggtttgatgtaagtaggttatcgtcgtgcgagtgtgtggatgcgatcgttcgagagattcggaggtaggtttgggatacgtttgagtgaatattttaggatatttttttggttagtatttgttttttagtgtttgtgatttagagtgggtatatgttgggagatagtaatgggtttgggtgtgtgtaaatgagtgtgatcggaagcgagtgtgagtttgatttaggtagggattatatagtattgttatatttgtttgttttttagtagaggattgaagtgcgggggtgggggtacggggtcggaatagaatgtttttgggatattttggtaaatagtagtcggaagtaaaggggtagttgtgtaaacg

SEQ ID NO: 3 - DNA, 965 bp, Artificial Sequence
  chemically treated genomic DNA (Homo sapiens)
cgtttgtatagttgtttttttgttttcggttgttgtttgttaagatgttttagagatattttatttcggtttcgtatttttattttcgtattttagttttttattaaagagtaggtaggtgtgatagtgttgtgtggtttttgtttagattaagtttatattcgttttcggttatatttatttatatatatttaaatttattattgttttttaatatgtgtttattttgaattatagatattaaaaaatagatattggttagaagagtattttgaggtgtttatttaaacgtgttttaagtttgttttcgagtttttcgggcggtcgtatttatatattcgtacgacgatgatttgtttgtattaaatttgtatatattacgaataggcgttttttattatatttaattacgcgtgattttaatagtttttatatttcgtagaatcgggttttagttttattgtgtttgagcgtttagtttagtagaggtagggaatcgggttttagttttattgtgtttgagcgtttagtttagtagaggtagggattaaggcgtttgtcgtttatagcgtttagagtggtaagaaagaagttttttagcgtttttattatttttcggtggaacgcgtagcgcgcgatttgttgtagtcggttcgggcgttgtttgtgcggtgagcgtagtagcggtatttcggggttgtgggcggaggtaaggattttagtttgaggttttttgaggtagagtagaaattagtttttaggtgtgtcgttcgtggcgcgagttacggaattttaggtatttcggggtcgtgttcgtatttttgtttttagtttttagaagcgtcgaaattttttagtacgatatttttgtttcgcgttatattttttttttttaggggttggtggttttcgtgtttaaaggaggtagcgggaacgtttaatttttaagatgtttttaattgatt

SEQ ID NO: 4 - DNA, 965 bp, Artificial Sequence
  chemically treated genomic DNA (Homo sapiens)
gattaattaagggtattttagaagttaggtgtttttgttgttttttttgagtatggaggttattaatttttagggggaagagatgtagtgtgaggtaggggtgttgtgttaagaaattttgatgtttttggggattgaggataaaggtgtggatatgattttggggtatttggagttttgtgatttgtgttatggatggtatatttaggggttaatttttgttttgttttaaagaattttaagttagagtttttgtttttgtttatagttttgggatgttgttgttgtgtttattgtataggtagtgtttggattggttgtagtagattgtgtgttgtgtgttttattgggagatggtggagatgttgaaaagttttttttttgttattttggatgttgtgggtggtaagtgttttagtttttatttttgttgagttgaatgtttaggtatagtggaattgaaatttggttttttatttttgttgagttgaatgtttaggtatagtggaattgaaatttggttttgtgggatgtgagagttgttgaggttatgtgtaattgggtgtgatggagggtgtttgtttgtgatgtgtgtaggtttgatgtaagtaggttattgttgtgtgagtgtgtggatgtgattgtttgagagatttggaggtaggtttgggatatgtttgagtgaatattttaggatatttttttggttagtatttgttttttagtgtttgtgatttagagtgggtatatgttgggagatagtaatgggtttgggtgtgtgtaaatgagtgtgattggaagtgagtgtgagtttgatttaggtagggattatatagtattgttatatttgtttgttttttagtagaggattgaagtgtgggggtgggggtatggggttggaatagaatgtttttgggatattttggtaaatagtagttggaagtaaaggggtagttgtgtaaatg

SEQ ID NO: 5 - DNA, 965 bp, Artificial Sequence
  chemically treated genomic DNA (Homo sapiens)
tgtttgtatagttgtttttttgtttttggttgttgtttgttaagatgttttagagatattttattttggttttgtatttttatttttgtattttagttttttattaaagagtaggtaggtgtgatagtgttgtgtggtttttgtttagattaagtttatatttgtttttggttatatttatttatatatatttaaatttattattgttttttaatatgtgtttattttgaattatagatattaaaaaatagatattggttagaagagtattttgaggtgtttatttaaatgtgttttaagtttgtttttgagttttttgggtggttgtatttatatatttgtatgatgatgatttgtttgtattaaatttgtatatattatgaataggtgttttttattatatttaattatgtgtgattttaatagtttttatattttgtagaattgggttttagttttattgtgtttgagtgtttagtttagtagaggtagggaattgggttttagttttattgtgtttgagtgtttagtttagtagaggtagggattaaggtgtttgttgtttatagtgtttagagtggtaagaaagaagttttttagtgtttttattattttttggtggaatgtgtagtgtgtgatttgttgtagttggtttgggtgttgtttgtgtggtgagtgtagtagtggtattttggggttgtgggtggaggtaaggattttagtttgaggttttttgaggtagagtagaaattagtttttaggtgtgttgtttgtggtgtgagttatggaattttaggtattttggggttgtgtttgtatttttgtttttagtttttagaagtgttgaaattttttagtatgatatttttgttttgtgttatattttttttttttaggggttggtggtttttgtgtttaaaggaggtagtgggaatgtttaatttttaagatgtttttaattgatt

SEQ ID NO: 6 - DNA, 21 bp, Artificial Sequence
  primer example 1
aggttatcgtcgtgcgagtgt

SEQ ID NO: 7 - DNA, 24 bp, Artificial Sequence
  primer example 1
tcactcaaacgtatcccaaaccta

SEQ ID NO: 8 - DNA, 25 bp, Artificial Sequence
  probe example 1
cgaatctctcgaacgatcgcatcca

SEQ ID NO: 9 - DNA, 25 bp, Artificial Sequence
  primer example 1 control
tggtgatggaggaggtttagtaagt

SEQ ID NO: 10 - DNA, 27 bp, Artificial Sequence
  primer example 1 control
aaccaataaaacctactcctcccttaa

SEQ ID NO: 11 - DNA, 30 bp, Artificial Sequence
  probe example 1 control
accaccacccaacacacaataacaaacaca

SEQ ID NO: 12 - DNA, 23 bp, Artificial Sequence
  primer example 2
ggatgtgagagttgttgaggtta

SEQ ID NO: 13 - DNA, 24 bp, Artificial Sequence
  primer example 2
acacacccaaacccattactatct

SEQ ID NO: 14 - DNA, 25 bp, Artificial Sequence
  probe example 2
acctccgaatctctcgaacgatcgc

SEQ ID NO: 15 - DNA, 28 bp, Artificial Sequence
  blocker example 2
tgttgaggttatgtgtaattgggtgtga

SEQ ID NO: 16 - DNA, 20 bp, Homo Sapiens
ccttagtccctacctctgct

SEQ ID NO: 17 - DNA, 21 bp, Homo Sapiens
ctcatttacacacacccaaac